(19) United States
(12) Patent Application Publication
(10) Pub. No.: US 2005/0038328 A1
(43) Pub. Date: Feb. 17, 2005

(54) Device for examining dysfunction of the bladder

(76) Inventors: Manfred Stoehrer, Muenchen (DE); Augustinus Kramer, Muenchen (DE); Guenther Kohout, Tiefenbach (DE)

Correspondence Address: JORDAN AND HAMBURG LLP, 122 EAST 42ND STREET, SUITE 4000, NEW YORK, NY, 10168, US

(21) Appl. No.: 10/494,174

(22) PCT Filed: Oct. 30, 2002
(86) PCT No.: PCT/EP02/12114

(51) Int. Cl. A61B 5/00

(52) U.S. Cl. 600/301

(57) ABSTRACT

Apparatus for providing data for facilitating diagnosis of impairments to bladder function in humans by measurement of parameters associated therewith, includes device(s) for measuring at least one somatic function, such as pulse, blood pressure, galvanic skin resistance, core body temperature, and skin temperature. Certain embodiments of the apparatus also include device(s) for measuring non-somatic functions, such as bladder pressure, rectal pressure, urethral pressure, anal sphincter pressure, incontinenance event occurrence, and incontinence event severity. Other embodiments further include devices for measuring one or more of a patient's body position, body movements, body angle, cough reflex response, and bladder fullness. Still other embodiments further include devices for recording and storing measured data; a video device for observing a patient while sleeping; and electronic data transmitting and receiving devices for remote data transfer. In one embodiment, sensors for all measuring devices are located on a patient-wearable module.

The present invention relates to apparatus for investigating pathological impairments to bladder functions.

Impairments to bladder functions due to damage to the central nervous system are the most common cause of urinary incontinence. Untreated, they always lead to a massive decline in quality of life and to substantially reduced life expectancy, especially after lesion of the spinal cord with paraplegia or other accident-related damage to the central nervous system, e.g., cranio-cerebral trauma. Thirty years ago, the survival time for quadriplegia, that is, paralysis of arms and legs, was a few months; for paraplegia, that is, paralysis of the legs only, it was a few years. At that time therapeutic measures in the urological field comprised placement of a permanent catheter or care with diapers or a urinal, as well as surgical measures, possibly in terms of performing a sphincterotomy or installation of an artificial urine drain by circumventing the bladder by means of a section of intestine, e.g., ileal conduit. Intensive neurological efforts over the past 20 years have led to a substantial improvement in this situation. Thus, today it can be assumed that a paraplegic has practically no reduction in life expectancy provided he undergoes regular check-ups in specialized centers. Even after quadriplegia, substantial improvements in terms of life expectancy have been achieved in the urological field consistent with the magnitude of the lesion.

Thus a number of conservative and surgical therapy techniques are known with which specific pathological findings can be successfully treated in a very targeted manner. It is necessary to diagnose the precise pathological findings in order to employ the correct, custom-designed therapy. However, in problematic cases this is not possible with the required certainty, so that frequently an incorrect or inadequate therapy is employed.

In standard urodynamics, the bladder is filled with a liquid using a catheter. A fill speed of 20 to 50 mL/min is provided, so that the filling process takes approximately five minutes, which is approximately 50 to 100 times faster than the bladder fills with urine naturally. While the bladder is undergoing artificial filling, the pressure inside the bladder and the pressure inside the rectum are measured using two pressure sensors attached to catheters. A contraction in the bladder musculature results in a positive difference between bladder pressure and rectum pressure. If this contraction occurs involuntarily while the bladder is being filled, this is an indication of a pathological change in bladder control. Rectum pressure is a reference value for bladder pressure. Because when there is coughing or when the abdominal musculature is tensed, the interior pressure in the lower body increases overall, so that one measurement of bladder pressure alone is not adequate for detecting an involuntary contraction of the bladder musculature. In addition, in standard urodynamics, liquid volume during urination is frequently measured by means of a receptacle. In order to increase accuracy, this measurement process is repeated one or two times, so that the measurement can last a total of 45 to 60 minutes. The measurement can even take up to two hours for neurogenic patients.

The disadvantage of standard urodynamics is the diagnosis' high susceptibility to error. Thus, when the patient moves or changes position, positive differences between bladder pressure and rectum pressure occur that have not been caused by involuntary contractions. In addition, the unnaturally rapid filling of the bladder provokes involuntary contractions in the bladder musculature, even in healthy patients. Therefore it does not provide an evaluation with reliable certitude. This susceptibility to error is frequently a substantial limitation in the diagnostic reliability of urodynamic investigations, especially in neurogenic patients.

Furthermore, in neurogenic patients with autonomic dysreflexia (high blood pressure, caused e.g. by movements or certain conditions of various organs such as stomach, intestinal tract, and urinary tract, due to insufficiency in blood pressure regulation using the nervus vagus), this autonomic dysreflexia is easily provoked by rapid bladder filling and/or the bladder contraction caused thereby.

In video urodynamics, in addition to standard urodynamics the patient is subjected to x-ray examination approximately 8 to 10 times during the measurement process. This is intended to make it possible to diagnose bladder/sphincter dyssynergia. Such dyssynergia is characterized in that the bladder musculature contracts although the sphincter musculature has not released the urethra. One possible consequence is that the urine backs up to the kidneys, which can suffer damage as a result.

It is therefore one object of the present invention to eliminate the aforesaid disadvantages of known apparatus for investigating pathological impairments to bladder functions. In particular known apparatus are to be further developed such that evaluation certitude, i.e., diagnostic certitude, is substantially increased.

This object is attained by an apparatus in accordance with claim 1. The dependent claims concern advantageous embodiments.

The present invention is based on the recognition that the investigation of somatic functions can be used to diagnose pathological impairments to bladder functions. It is therefore inventively suggested to provide one or a plurality of means for measuring one or a plurality of somatic functions. These functions are in particular pulse, blood pressure, skin resistance, body core temperature and/or skin temperature of the person being examined. It has been demonstrated that evaluating one or a plurality of these somatic functions can be adequate for being able to diagnose pathological impairments to bladder functions, since somatic functions have a direct correlation to bladder contraction. In addition, there is a correlation between somatic functions and bladder filling.

A bladder contraction is triggered by the somatic system. As the consequence of such triggering, the sweat glands open so that shortly prior to and during a bladder contraction the skin resistance drops almost to zero, similar to a short circuit. Since normally skin resistance—depending on the position of the measuring electrodes—is in the range of several kiloohms, this effect can be measured with great reliability.

Due to somatic control for triggering a bladder contraction, a contraction of the blood vessels occurs, as well, and as a result there is an immediate increase in blood pressure, which can itself be measured with great reliability. The pulse or cardiac rate increases, as well, as an indirect result of the increase in blood pressure and due to the direct somatic control. Then, body core temperature drops as a result of the increased pulse rate, since increased blood circulation effects an elevated cooling effect in the interior of the body.

With an inventive apparatus, measurement is performed during a natural filling of the bladder by the patient's kidneys, diuresis. This avoids provoking autonomic dysreflexia due to unnaturally rapid filling of the bladder.

In addition, in accordance with the present invention means for measuring non-somatic functions can be provided, in particular for measuring bladder pressure, rectum pressure, urethral pressure, and/or anal sphincter pressure. These measurements diagnose impairments to bladder functions as is commonly done in the conventional manner in standard urodynamics.

Finally, in accordance with the present invention additional means can be provided for measuring non-somatic functions or parameters, in particular for measuring the position of the patient, his movements during the investigation, the angle of his legs or thighs relative to his upper body, for measuring coughing, how full his bladder is, and tensing in the abdominal musculature and the musculature of the urethral sphincter muscle. All of these functions or parameters can be important for involuntary contractions of the bladder musculature.

The result is that the certitude of the evaluation increases if there is any uncertainty when the data from just somatic functions are evaluated.

The apparatus for providing data for facilitating diagnosis of impairments to bladder function in a human by enabling measurement of parameters associated with bladder function, according to the present invention, generally includes at least one somatic function measuring means for measuring a somatic function of a human. The somatic function measuring means is selected from the group consisting of: pulse measuring means; blood pressure measuring means; galvanic skin resistance measuring means; core body temperature measuring means; and skin temperature measuring means.

In the above apparatus, when the at least one somatic function measuring means is pulse measuring means, the pulse measuring means includes at least one electrode cable and at least one pulse measuring clip selected from the group consisting of ear-attachable pulse measuring clips; finger-attachable pulse measuring clips; and, where there is a plurality of pulse measuring clips, combinations thereof.

In the foregoing apparatus, having blood pressure measuring means, it is preferable that the blood pressure measuring means is non-invasive.

In the general apparatus of the present invention, when the at least one somatic function measuring means is galvanic skin resistance measuring means, the galvanic skin resistance measuring means includes an electrical voltage measuring apparatus that measures an electrical voltage from an applied electrical voltage source at at least two sites on a skin surface; at least one electrode cable; and a plurality of electrodes.

In the foregoing apparatus, having core body temperature measuring means, the core body temperature measuring means is one of a rectally insertable temperature sensor for measuring core body temperature; and an orally ingestible temperature sensor for measuring core body temperature with an electronic signal transmitter for transmitting an electronic signal representative of core body temperature measured by the temperature sensor, to an external electronic signal receiver.

In the general apparatus of the present invention, when the at least one somatic function measuring means is skin temperature measuring means, the skin temperature measuring means includes a skin surface contacting temperature sensor.

The foregoing general apparatus of the present invention, including at least one, and up to all of the aforesaid somatic function measuring means may further include at least one non-somatic function measuring means for measuring a non-somatic function of a human. The non-somatic function measuring means is selected from the group consisting of: bladder pressure measuring means; rectal pressure measuring means; urethral pressure measuring means; anal sphincter pressure measuring means; incontinenance event occurrence measuring means; and incontinence event severity measuring means.

In an embodiment of the general apparatus of the present invention that also includes non-somatic function measuring means, when the at least one non-somatic function measuring means is bladder pressure measuring means, the bladder pressure measuring means includes a catheter having a pressure sensor thereon for measuring bladder pressure.

In the foregoing apparatus, wherein the pressure sensor is on a distal end of the catheter, such that when the catheter is trans urethrally inserted into a bladder, the sensor measures the bladder pressure. The bladder pressure measuring means further includes an electronic signal transmitter for transmitting an electronic signal representative of the measured bladder pressure.

In the foregoing apparatus, the pressure sensor is at a proximal end of the catheter, on an external, non-urethrally insertable part thereof, and the bladder pressure measuring means further includes a pressure detector/transmitter on a distal end of the catheter, such that when the catheter is transurethrally inserted into a bladder, the pressure detector/transmitter detects bladder pressure and transmits the bladder pressure through the catheter to the pressure sensor.

In an embodiment of the general apparatus of the present invention that also includes non-somatic function measuring means, when the at least one non-somatic function measuring means is rectal pressure measuring means, the rectal pressure measuring means includes a catheter having a pressure sensor thereon for measuring rectal pressure.

In the foregoing apparatus, the pressure sensor is on a distal end of the catheter, such that when the catheter is rectally inserted, the sensor measures the rectal pressure. The rectal pressure measuring means further includes an electronic signal transmitter for transmitting an electronic signal representative of the measured rectal pressure.

In the foregoing apparatus, the pressure sensor is at a proximal end of the catheter, on an external, non-rectally insertable part thereof, and the rectal pressure measuring means further includes a pressure detector/transmitter on a distal end of the catheter, such that when the catheter is rectally inserted, the pressure detector/transmitter detects rectal pressure and transmits the rectal pressure through the catheter to the pressure sensor.

In an embodiment of the general apparatus of the present invention that also includes non-somatic function measuring means, when the at least one non-somatic function measuring means is urethral pressure measuring means, the urethral pressure measuring means includes a catheter having a pressure sensor thereon for measuring urethral pressure.

In the foregoing apparatus, the pressure sensor is on a distal end of the catheter, such that when the catheter is urethrally inserted, the sensor measures the urethral pressure; and the urethral pressure measuring means further includes an electronic signal transmitter for transmitting an electronic signal representative of the measured urethral pressure.

In the foregoing apparatus, the pressure sensor is at a proximal end of the catheter, on an external, non-urethrally insertable part thereof, and the urethral pressure measuring means further includes a pressure detector/transmitter on a distal end of the catheter, such that when the catheter is urethrally inserted, the pressure detector/transmitter detects urethral pressure and transmits the urethral pressure through the catheter to the pressure sensor.

In an embodiment of the general apparatus of the present invention that also includes non-somatic function measuring means, when the at least one non-somatic function measuring means is anal sphincter pressure measuring means, the anal sphincter pressure measuring means includes a catheter having a pressure sensor thereon for measuring anal sphincter pressure.

In the foregoing apparatus, the pressure sensor is on a distal end of the catheter, such that when the catheter is rectally inserted up to an anal sphincter, the sensor measures the anal sphincter pressure. The anal sphincter pressure measuring means further includes an electronic signal transmitter for transmitting an electronic signal representative of the measured anal sphincter pressure.

In the foregoing apparatus, the pressure sensor is at a proximal end of the catheter, on an external, non-rectally insertable part thereof, and the anal sphincter pressure measuring means further includes a pressure detector/transmitter on a distal end of the catheter, such that when the catheter is rectally inserted up to an anal sphincter, the pressure detector/transmitter detects anal sphincter pressure and transmits the anal sphincter pressure through the catheter to the pressure sensor.

One embodiment of the apparatus of the present invention includes both bladder pressure measuring means and urethral pressure measuring means. Preferably, this embodiment has a single dual lumen catheter with at least one pressure sensor thereon for measuring bladder pressure and for measuring urethral pressure.

Another embodiment of the apparatus of the present invention includes rectal pressure measuring means and anal sphincter pressure measuring means. This embodiment has a single catheter with at least one pressure sensor thereon for measuring rectal pressure and for measuring anal sphincter pressure.

In an embodiment of the general apparatus of the present invention that also includes non-somatic function measuring means, when the at least one non-somatic function measuring means is incontinenance event occurrence measuring means, the incontinenance event occurrence measuring means includes a moisture sensor.

In the foregoing apparatus, the moisture sensor utilizes at least one of resistance measurement and capacity measurement. The moisture sensor is preferably integrated into a diaper.

In another embodiment of the general apparatus of the present invention that also includes non-somatic function measuring means, when the at least one non-somatic function measuring means is incontinenance event occurrence measuring means, the incontinenance event occurrence measuring means includes a flowmeter that is alternatively externally and internally patient-borne.

In the foregoing apparatus, the flowmeter is preferably on a catheter.

In an embodiment of the general apparatus of the present invention that also includes non-somatic function measuring means, when the at least one non-somatic function measuring means is incontinenance event severity measuring means, the incontinenance event severity measuring means includes a diaper and a moisture sensor.

One embodiment of the apparatus of the present invention includes bladder pressure measuring means and incontinenance measuring means. Preferably, this embodiment has a single catheter having a pressure sensor thereon for measuring rectal pressure and for bearing a flowmeter for measuring incontinenance.

A general embodiment of the apparatus of the present invention that includes at least one somatic function measuring means and additionally may include at least one non-somatic function measuring means, may further include body position measuring means for measuring a position of a body of a patient.

In the foregoing apparatus, the body position measuring means includes a hollow outer sphere and an inner sphere, rotatably borne within the hollow outer sphere, the inner sphere having at least one of a surface segment thereof removed; and a center of gravity different from a geometric center of the inner sphere, such that the inner sphere maintains a constant angle relative to a gravitational direction.

In this embodiment of the apparatus, the inner sphere of the body position measuring means has electrical contact surfaces on an external surface thereof, and the outer sphere of the body position measuring means has electrical contact surfaces on an internal surface thereof, such that a position of a body is determinable when the electrical contact surfaces on the internal surface of the outer sphere are in electrical contact with the electrical contact surfaces on the external surface of the inner sphere.

A general embodiment of the apparatus according to the present invention that includes any of the foregoing described measuring means may also further include body movement measuring means for measuring movement of a body of a patient.

In the foregoing apparatus, the body movement measuring means includes a shock sensor attachable to a body of a patient.

A general embodiment of the apparatus according to the present invention that includes any of the foregoing described measuring means may also further include body angle measuring means for measuring a body angle between an upper body and a lower extremity, selected from the group consisting of: a thigh; and a leg, of the patient.

In the foregoing apparatus, the body angle measuring means includes at least one light guide, longitudinally attachable to the lower extremity of the patient, for emitting light energy; a light source for providing light energy to each of the at least one light guide; a light sensor, for detecting light energy emitted by each of the at least one light guide, and for producing a signal representative thereof; and calculation means for calculating the body angle from the signal produced by the light sensor.

In the foregoing apparatus, each of the at least one light guide preferably includes a fiber optic cable.

A general embodiment of the apparatus according to the present invention that includes any of the foregoing described measuring means may also further include cough detection means for detecting and registering the occurrence of a cough reflex in the patient.

In the foregoing apparatus, the cough detection means includes acoustic detection means for detecting acoustic signals.

According to one embodiment of the foregoing apparatus, the acoustic detection means is a microphone.

According to another embodiment of the foregoing apparatus, the cough detection means includes a longitudinally elastically extensible thoracic band for close contacting placement around a thorax of a patient; and a sensor for measuring a change in length of the thoracic band upon occurrence of a cough reflex in the patient.

An embodiment of the foregoing apparatus that includes acoustic detection means further includes a microprocessor for analyzing the acoustic signals detected by the acoustic detection means for determining the occurrence of a cough reflex in the patient.

A general embodiment of the apparatus according to the present invention that includes any of the foregoing described measuring means may also further include muscle tension measuring means for measuring muscle tension in at least one muscle selected from the group consisting of: abdominal musculature; and urethral sphincter muscle; the muscle tension measuring means including a plurality of electrodes for measuring voltage produced by the muscle.

A general embodiment of the apparatus according to the present invention that includes any of the foregoing described measuring means may also further include bladder fullness measuring means for measuring a volume of fluid in a bladder of a patient.

A general embodiment of the apparatus according to the present invention that includes any of the foregoing described measuring means may also further include data recording and storing means for recording and storing data obtained by any of the measuring means and the detection means.

In the foregoing apparatus, the data recording and storing means stores data for a predetermined period of time.

In the foregoing apparatus, the data recording and storing means includes at least one of: magnetic medium; optical medium; analog medium; digital medium; and combinations thereof.

In the foregoing apparatus, when the recording and storing means is a digital medium, the digital medium includes a plurality of digital integrated circuit memory chips.

In any of the foregoing embodiments of the apparatus having recording and storing means, the recording and storing means is preferably erasable and rewriteable.

In any of the foregoing embodiments of the apparatus having recording and storing means, the recording and storing means has sufficient capacity to record and store data for at least two hours, preferably for longer than two hours, more preferably for six hours, and most preferably for 24 hours.

A general embodiment of the apparatus according to the present invention that includes any of the foregoing described measuring means may also further include marker means for enabling patient-actuated manual entry of data into the recording and storing means.

In the foregoing apparatus, the data to be manually entered is data relating to occurrence of patient-subjectively-experienced events relating to bladder function.

In the foregoing apparatus, the marker means is alternatively one or a plurality of user-actuated devices. The user-actuated devices are selected from the group consisting of: buttons, switches, knobs, and, where there is a plurality of the user-actuated devices, combinations thereof.

In the foregoing apparatus, when there is one user-actuated device, the device is multifunctional and capable of separately marking occurrence of a plurality of different events of a predetermined nature; and when there is a plurality of user-actuated devices, each of the devices is dedicated to marking single or repetitive occurrence of an event of a predetermined nature.

In the foregoing apparatus, the marker means is capable of determining a relative intensity of each occurrence of the patient-subjectively-experienced event relating to bladder function triggering user actuation of the marker means.

The relative intensity of each the occurrence of the patient-subjectively-experienced event relating to bladder function is expressed through a characteristic parameter of the marker means.

According to one embodiment of the foregoing apparatus, the marker means is a variable rheostat and characteristic parameter is electrical resistance.

According to certain embodiments of the foregoing apparatus, a parameter of an output signal of the marker means is proportional to a relative intensity of the patient-subjectively-experienced event relating to bladder function.

In embodiments of the foregoing apparatus, when the marker means includes a button, relative intensity of the patient-subjectively-experienced event relating to bladder function is expressed by one of proportionality of a user-applied force to the button and frequency of repetitive actuation of the button; when the marker means includes a switch, relative intensity of the patient-subjectively-experienced event relating to bladder function is expressed by one of selection of a position of a more than bipositional multipositional switch and by frequency of repetitive actuation of the switch; and when the marker means includes a rotatable knob, relative intensity of the patient-subjectively-experienced event relating to bladder function is expressed by selection of a position of the knob coordinated to indicia of intensity.

In any of the foregoing embodiments of the apparatus including marker means, some of the patient-subjectively-experienced events relating to bladder function include, but are not limited to: consumption of liquid by the patient; sensation of an urge to urinate; and an actual urination event.

A general embodiment of the apparatus according to the present invention that includes any of the foregoing described measuring means may also further include video image recording means for recording patient image data.

In the foregoing apparatus, the video image recording means can include, but is not limited to, one selected from the group consisting of a video camera and a digital camera.

In the foregoing apparatus, one main function of the video image recording means is to record patient image data, while the patient is sleeping, so as to determine occurrence of events symptomatic of impaired bladder function in the patient, such as movement and restlessness, which may signify pain or discomfort, and/or to record the time of occurrence of an actual event, related to bladder impairment, such as an incontinenance event, which occurs while the patient is in a somnolent state and may not recall or be able to precisely time the occurrence of later when the patient is awake.

In the foregoing apparatus, when the video image recording means is a digital camera, the digital camera further includes digital storage means for storing digital image data recorded by the digital camera for a period of up to 12 hours. The digital storage means includes at least one digital memory card.

A general embodiment of the apparatus according to the present invention that includes any of the foregoing described measuring means may also further include a transmitter for transmitting data representative of measured values obtained by at least some of the measuring means and the detection means.

In the foregoing apparatus, preferably the transmitter is electronic and the data is transmitted electronically.

A general embodiment of the apparatus according to the present invention that includes any of the foregoing described measuring means may also further include a receiver for receiving data transmitted by the transmitter.

In the foregoing apparatus, preferably the receiver is electronic and the data is transmitted by the transmitter and received by the receiver electronically.

In the foregoing apparatus, the data is electronically transmitted and received by one selected from the group consisting of: radio waves; and light waves, but is not limited thereto.

According to certain embodiments of the foregoing apparatus, the data is electronically transmitted and received by radio waves.

In certain embodiments of the foregoing apparatus, electronic data transmission and reception is wireless.

In one preferred embodiment, thereof, the radio waves are short range radio waves. One example of such technology is that incorporating what is known as Bluetooth® technology.

In certain embodiments of the foregoing apparatus, at least one of electronic data transmission by the transmitter; electronic data reception by the receiver; and electronic communication between the transmitter and the receiver, occurs automatically.

In one embodiment of the foregoing apparatus, the data recording and storing means and the receiver are proximally located.

In yet another general embodiment of the apparatus according to the present invention that includes any of the foregoing described measuring means and detection means, the data recording and storing means is housed in a portable module. In certain embodiments thereof, the portable module is patient-wearable. Preferably, all of the measuring means and the detection means are in electronic communication with the module. For convenience and mobility, the module is battery powered.

The present invention is explained in greater detail in the following using a preferred exemplary embodiment and the accompanying drawing figures, wherein:

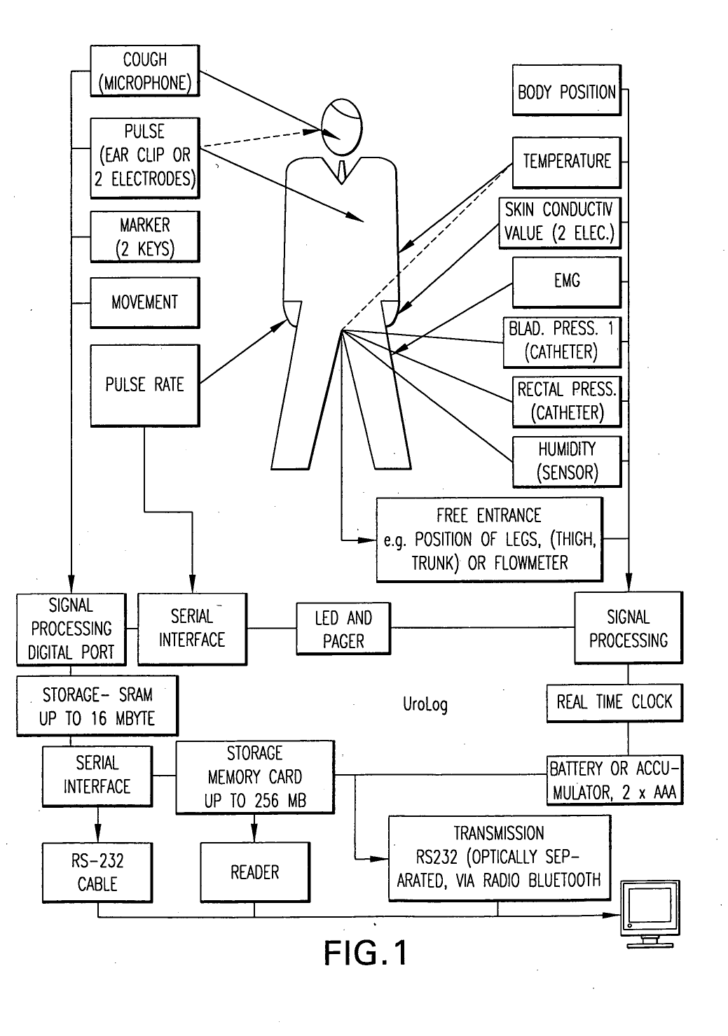

FIG. 1 illustrates a block diagram of a preferred embodiment of the inventive apparatus (“UroLog”), and

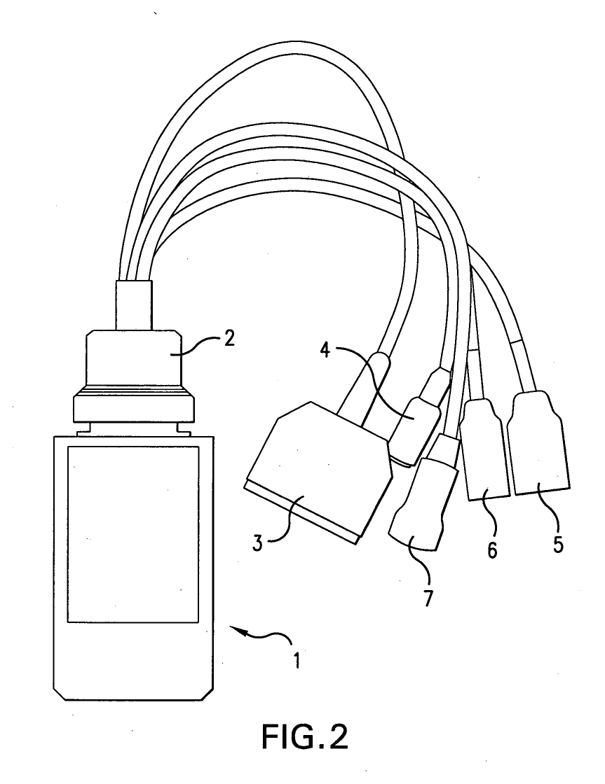

FIG. 2 illustrates one module in accordance with a preferred embodiment of the inventive apparatus (“UroLog”) with connectors and connecting cables.

In accordance with one preferred embodiment, the inventive apparatus for investigating impairments to bladder functions includes a compact module that during the investigation period is situated on or in the vicinity of the body of the person being examined. FIG. 1 illustrates this module symbolically. Connected to the module is means or a plurality of means for measuring various somatic and/or non-somatic functions or parameters of the person via serial interfaces or via signal preparation units. If necessary, one or more other means for measuring other conditions can also be connected to the module.

The data measured by these means are stored by means for recording data that is arranged in the module and preferably includes 4 MBits or 8 MBits of memory. However, a transmitter can also be arranged in or on the module; it transmits the data to a stationary receiver. In this case, the means for recording data does not have to be in arranged in the module, but can also be arranged stationary at the receiver, whereby the receiver forwards the data to the recording means for recording.

As in conventional urodynamics, the apparatus illustrated in FIG. 1 measures bladder pressure and rectal pressure. Pressure sensors can be employed that are introduced into the patient's bladder or rectum via a catheter. A Wheatstone bridge can be used for the pressure sensors, whereby a resistance depends on pressure. The pressure sensors are connected to the module via electrically conducting cable.

In the apparatus illustrated in FIG. 1, pulse rate, skin resistance, and skin temperature are measured for the somatic functions. When there is a contraction in the bladder musculature—depending on the position of the measuring electrodes—the skin resistance drops from a few kiloohms to almost zero, similar to a short circuit. Two or more electrodes can be used for this that can be arranged e.g. in the region of the hand. As another result of a contraction of the bladder musculature, blood vessels constrict so that blood pressure also rises immediately. Blood pressure can be measured preferably in a non-invasive manner.

The pulse rate also increases as a result of the increase in blood pressure and due to direct somatic triggering. In the apparatus illustrated in FIG. 1, the pulse rate is preferably measured using an ear clip. With such an ear clip, the earlobe is illuminated with a light source, whereby the quantity of light that passes through the ear changes periodically with the pulse beat. A light sensor measures this periodic change in light intensity. Alternatively, two electrodes that are arranged in the region of the heart can also be employed to measure the pulse. These electrodes can measure a voltage that is produced during contraction of the cardiac musculature. In addition, it is possible to measure the pulse using a finger clip, whereby oxygen saturation provides the initial value for the measurement.

Since somatic triggering opens the sweat glands during a contraction of the bladder musculature, there is elevated evaporation from the surface of the skin so that the skin temperature drops correspondingly. This effect can be measured using temperature-sensitive resistances that can be arranged on the skin surface, e.g. in the region of an arm.

A contraction of the bladder musculature or a positive difference between bladder pressure and rectum pressure can also result from corresponding movements by the patient, e.g. tensing of the abdominal musculature, or from coughing. Therefore in accordance with one preferred embodiment of the invention, a sensor for measuring tensing of the abdominal musculature, a motion sensor, and a sensor for determining body position are provided. Coughing, which is connected to sudden tensing of the abdominal musculature, can be detected using a microphone. Alternatively, however, a band can also be arranged about the thorax, whereby coughing is detected via sudden shortening of the circumference of the thorax.

In addition to being important for bladder filling, the patient's movement, body position, and leg position during the investigation period is important for the urge to urinate and for involuntary contractions of the bladder musculature. Leg position, in particular the angle between upper body and thigh, can be measured using a fiberglass cluster, whereby a light source is arranged at one end of the fiberglass cluster, and a light sensor is arranged at the other end. The fiberglass cluster is preferably attached laterally on the thorax and laterally along the thigh. When the patient is standing or lying flat, a maximum quantity of light travels through the fiberglass cluster. However, when the angle between upper body and thigh changes, the fiberglass cluster is bent somewhat so that less light travels all the way through the fiberglass cluster.

The inventive apparatus can furthermore include a video or digital cameral and a memory card for collecting image data. Image data about movements can be collected, especially while the patient is sleeping, in order to attain certitude in the evaluation.

Finally, a moisture sensor that is arranged preferably in a diaper and a flow measuring device can measure the existence and severity of any incontinence.

Marking buttons for manual actuation by the patient can also be provided. This makes it possible for the patient to mark certain events, such as e.g. the sensation of a need to urinate. Corresponding inputs can later also be taken into account in the evaluation.

The module is preferably operated using a battery or an accumulator, in particular two AAA batteries. This makes it possible for the person to be evaluated to move freely, as he normally would, for the period of the investigation, which can take up to 30 hours, or, depending on scanning rate and memory, up to 20 days. The scanning rate can be selected freely starting with scanning every two minutes up to a maximum 1024 Hz.

After the investigation period, the module can be connected via a serial interface, in particular via an RS-232 port with interface, to a computer with which the measured data can be evaluated using evaluation software. Evaluation of the data can also be evaluated by a physician using a graphic representation, however, in order to arrive at the correct diagnosis.

FIG. 2 illustrates a module 1 in accordance with one preferred embodiment of the inventive apparatus (“UroLog”) with connectors and cables. The module 1 is 58 mm×105 mm×19 mm in size, weighs approximately 0.2 kg, and is splash-resistant or—if necessary—water-tight. Attached to the module is a connector 2 with cables, whereby at the ends of the cables are an “EMG, ECG, EDA” electrode connection box 3, a connector 4 for a microphone, a connenctor 5 for a catheter for measuring rectal pressure, a connector 6 for a catheter for measuring bladder pressure, and a connector 7 for a temperature sensor.

Listing of reference numerals used in the drawing figures and elements of the apparatus of the present invention denoted thereby:
1. Coughing (microphone)
2. Pulse (ear clip or 2 electrodes)
3. Marker (2 buttons)
4. Movement
5. Pulse, oxygen saturation (finger clip)
6. Signal preparation, digital port
7. Serial interface
8. Memory, SRAM up to 16 MBytes
9. Serial interface
10. Memory, 256 MB memory card
11. RS-232 cable
12. Reader
13. Body position
14. Temperature
15. Skin measurement (2 electrodes)
16. EMG
17. Bladder pressure 1 (catheter)
18. Rectal pressure (catheter)
19. Moisture (sensor)
20. Free access e.g. leg position (thigh—thorax) or flow meter
21. LED and beeper
22. Signal preparation, 10 Bit ADC
23. Real time clock
24. Battery or accumulator, 2×AAA
25. RS232 transmission (optical separation) by radio, bluetooth

1-63. (Canceled).

64. Apparatus for providing data for facilitating diagnosis of impairments to bladder function in a human by enabling measurement of parameters associated with bladder function, said apparatus comprising at least one somatic function measuring means for measuring a somatic function of a human, said somatic function measuring means selected from the group consisting of: pulse measuring means; blood pressure measuring means; galvanic skin resistance measuring means; core body temperature measuring means; and skin temperature measuring means.

65. Apparatus according to claim 64, wherein when said at least one somatic function measuring means is pulse measuring means, said pulse measuring means comprises at least one electrode cable and at least one pulse measuring clip selected from the group consisting of ear-attachable pulse measuring clips; finger-attachable pulse measuring clips; and, where there is a plurality of said pulse measuring clips, combinations thereof.

66. Apparatus according to claim 64, wherein when said at least one somatic function measuring means is blood pressure measuring means, said blood pressure measuring means is non-invasive.

67. Apparatus according to claim 64, wherein when said at least one somatic function measuring means is galvanic skin resistance measuring means, said galvanic skin resistance measuring means comprises: an electrical voltage measuring apparatus that measures an electrical voltage from an applied electrical voltage source at at least two sites on a skin surface; at least one electrode cable; and a plurality of electrodes.

68. Apparatus according to claim 64, wherein when said at least one somatic function measuring means is core body temperature measuring means, said core body temperature measuring means comprises one of: a rectally insertable temperature sensor for measuring core body temperature; and an orally ingestible temperature sensor for measuring core body temperature with an electronic signal transmitter for transmitting an electronic signal representative of core body temperature measured by said temperature sensor, to an external electronic signal receiver.

69. Apparatus according to claim 64, wherein when said at least one somatic function measuring means is skin temperature measuring means, said skin temperature measuring means comprises a skin surface contacting temperature sensor.

70. Apparatus according to claim 64, further comprising at least one non-somatic function measuring means for measuring a non-somatic function of a human, said non-somatic function measuring means selected from the group consisting of: bladder pressure measuring means; rectal pressure measuring means; urethral pressure measuring means; anal sphincter pressure measuring means; incontinenance event occurrence measuring means; and incontinence event severity measuring means.

71. Apparatus according to claim 70, wherein when said at least one non-somatic function measuring means is bladder pressure measuring means, said bladder pressure measuring means comprises a catheter having a pressure sensor thereon for measuring bladder pressure.

72. Apparatus according to claim 71, wherein said pressure sensor is on a distal end of said catheter, such that when said catheter is trans urethrally inserted into a bladder, said sensor measures said bladder pressure; and said bladder pressure measuring means further comprises an electronic signal transmitter for transmitting an electronic signal representative of said measured bladder pressure.

73. Apparatus according to claim 71, wherein said pressure sensor is at a proximal end of said catheter, on an external, non-urethrally insertable part thereof, and said bladder pressure measuring means further comprises a pressure detector/transmitter on a distal end of said catheter, such that when said catheter is transurethrally inserted into a bladder, said pressure detector/transmitter detects bladder pressure and transmits said bladder pressure through said catheter to said pressure sensor.

74. Apparatus according to claim 70, wherein when said at least one non-somatic function measuring means is rectal pressure measuring means, said rectal pressure measuring means comprises a catheter having a pressure sensor thereon for measuring rectal pressure.

75. Apparatus according to claim 74, wherein said pressure sensor is on a distal end of said catheter, such that when said catheter is rectally inserted, said sensor measures said rectal pressure; and said rectal pressure measuring means further comprises an electronic signal transmitter for transmitting an electronic signal representative of said measured rectal pressure.

76. Apparatus according to claim 74, wherein said pressure sensor is at a proximal end of said catheter, on an external, non-rectally insertable part thereof, and said rectal pressure measuring means further comprises a pressure detector/transmitter on a distal end of said catheter, such that when said catheter is rectally inserted, said pressure detector/transmitter detects rectal pressure and transmits said rectal pressure through said catheter to said pressure sensor.

77. Apparatus according to claim 70, wherein when said at least one non-somatic function measuring means is urethral pressure measuring means, said urethral pressure measuring means comprises a catheter having a pressure sensor thereon for measuring urethral pressure.

78. Apparatus according to claim 77, wherein said pressure sensor is on a distal end of said catheter, such that when said catheter is urethrally inserted, said sensor measures said urethral pressure; and said urethral pressure measuring means further comprises an electronic signal transmitter for transmitting an electronic signal representative of said measured urethral pressure.

79. Apparatus according to claim 77, wherein said pressure sensor is at a proximal end of said catheter, on an external, non-urethrally insertable part thereof, and said urethral pressure measuring means further comprises a pressure detector/transmitter on a distal end of said catheter, such that when said catheter is urethrally inserted, said pressure detector/transmitter detects urethral pressure and transmits said urethral pressure through said catheter to said pressure sensor.

80. Apparatus according to claim 70, wherein when said at least one non-somatic function measuring means is anal sphincter pressure measuring means, said anal sphincter pressure measuring means comprises a catheter having a pressure sensor thereon for measuring anal sphincter pressure.

81. Apparatus according to claim 80, wherein said pressure sensor is on a distal end of said catheter, such that when said catheter is rectally inserted up to an anal sphincter, said sensor measures said anal sphincter pressure; and said anal sphincter pressure measuring means further comprises an electronic signal transmitter for transmitting an electronic signal representative of said measured anal sphincter pressure.

82. Apparatus according to claim 80, wherein said pressure sensor is at a proximal end of said catheter, on an external, non-rectally insertable part thereof, and said anal sphincter pressure measuring means further comprises a pressure detector/transmitter on a distal end of said catheter, such that when said catheter is rectally inserted up to an anal sphincter, said pressure detector/transmitter detects anal sphincter pressure and transmits said anal sphincter pressure through said catheter to said pressure sensor.

83. Apparatus according to claim 70, comprising bladder pressure measuring means and urethral pressure measuring means; and comprising a single dual lumen catheter having at least one pressure sensor thereon for measuring bladder pressure and for measuring urethral pressure.

84. Apparatus according to claim 70, comprising rectal pressure measuring means and anal sphincter pressure measuring means; and comprising a single catheter having at least one pressure sensor thereon for measuring rectal pressure and for measuring anal sphincter pressure.

85. Apparatus according to claim 70, wherein when said at least one non-somatic function measuring means is incontinenance event occurrence measuring means, said incontinenance event occurrence measuring means comprises a moisture sensor.

86. Apparatus according to claim 85, wherein said moisture sensor utilizes at least one of resistance measurement and capacity measurement; and said moisture sensor is integrated into a diaper.

87. Apparatus according to claim 70, wherein when said at least one non-somatic function measuring means is incontinenance event occurrence measuring means, said incontinenance event occurrence measuring means comprises a flowmeter that is alternatively externally and internally patient-borne.

88. Apparatus according to claim 87, wherein said flowmeter is on a catheter.

89. Apparatus according to claim 70, wherein when said at least one non-somatic function measuring means is incontinenance event severity measuring means, said incontinenance event severity measuring means comprises a diaper and a moisture sensor.

90. Apparatus according to claim 70, comprising bladder pressure measuring means and incontinenance measuring means; and comprising a single catheter having a pressure sensor thereon for measuring rectal pressure and for bearing a flowmeter for measuring incontinenance.

91. Apparatus according to claim 70, further comprising body position measuring means for measuring a position of a body of a patient.

92. Apparatus according to claim 91, wherein said body position measuring means comprises a hollow outer sphere and an inner sphere, rotatably borne within said hollow outer sphere, said inner sphere having at least one of: a surface segment thereof removed; and a center of gravity different from a geometric center of said inner sphere, such that said inner sphere maintains a constant angle relative to a gravitational direction.

93. Apparatus according to claim 92, wherein said inner sphere of said body position measuring means has electrical contact surfaces on an external surface thereof; and said outer sphere of said body position measuring means has electrical contact surfaces on an internal surface thereof, such that a position of a body is determinable when said electrical contact surfaces on said internal surface of said outer sphere are in electrical contact with said electrical contact surfaces on said external surface of said inner sphere.

94. Apparatus according to claim 91, further comprising body movement measuring means for measuring movement of a body of a patient.

95. Apparatus according to claim 94, wherein said body movement measuring means comprises a shock sensor attachable to a body of a patient.

96. Apparatus according to claim 94, further comprising body angle measuring means for measuring a body angle between an upper body and a lower extremity, selected from the group consisting of: a thigh; and a leg, of said patient.

97. Apparatus according to claim 96, wherein said body angle measuring means comprises at least one light guide, longitudinally attachable to said lower extremity of said patient, for emitting light energy; a light source for providing light energy to each of said at least one light guide; a light sensor, for detecting light energy emitted by each of said at least one light guide, and for producing a signal representative thereof; and calculation means for calculating said body angle from said signal produced by said light sensor.

98. Apparatus according to claim 97, wherein each of said at least one light guide comprises a fiber optic cable.

99. Apparatus according to claim 96, further comprising cough detection means for detecting and registering the occurrence of a cough reflex in said patient.

100. Apparatus according to claim 99, wherein said cough detection means comprises acoustic detection means for detecting acoustic signals.

101. Apparatus according to claim 100, wherein said acoustic detection means is a microphone.

102. Apparatus according to claim 99, wherein said cough detection means comprises a longitudinally elastically extensible thoracic band for close contacting placement around a thorax of a patient; and a sensor for measuring a change in length of said thoracic band upon occurrence of a cough reflex in said patient.

103. Apparatus according to claim 100, further comprising a microprocessor for analyzing said acoustic signals detected by said acoustic detection means for determining the occurrence of a cough reflex in said patient.

104. Apparatus according to claim 96, further comprising muscle tension measuring means for measuring muscle tension in at least one muscle selected from the group consisting of: abdominal musculature; and urethral sphincter muscle; said muscle tension measuring means comprising a plurality of electrodes for measuring voltage produced by said muscle.

105. Apparatus according to claim 104, further comprising bladder fullness measuring means for measuring a volume of fluid in a bladder of a patient.

106. Apparatus according to claim 105, further comprising data recording and storing means for recording and storing data obtained by any of said measuring means and said detection means.

107. Apparatus according to claim 106, wherein said data recording and storing means stores data for a predetermined period of time.

108. Apparatus according to claim 106, wherein said data recording and storing means comprises at least one of: magnetic medium; optical medium; analog medium; digital medium; and combinations thereof.

109. Apparatus according to claim 108, wherein when said recording and storing means is a digital medium, said digital medium comprises a plurality of digital integrated circuit memory chips.

110. Apparatus according to claim 106, wherein said recording and storing means is erasable and rewriteable.

111. Apparatus according to claim 106, wherein said recording and storing means has sufficient capacity to record and store data for at least two hours.

112. Apparatus according to claim 111, wherein said recording and storing means has sufficient capacity to record and store data for greater than two hours.

113. Apparatus according to claim 112, wherein said recording and storing means has sufficient capacity to record and store data for six hours.

114. Apparatus according to claim 112, wherein said recording and storing means has sufficient capacity to record and store data for 24 hours.

115. Apparatus according to claim 106, further comprising marker means for enabling patient-actuated manual entry of data into said recording and storing means.

116. Apparatus according to claim 115, wherein said data to be manually entered is data relating to occurrence of patient-subjectively-experienced events relating to bladder function.

117. Apparatus according to claim 115, wherein said marker means is alternatively one or a plurality of user-actuated devices.

118. Apparatrus according to claim 117, wherein said user-actuated devices are selected from the group consisting of: buttons, switches, knobs, and, where there is a plurality of said user-actuated devices, combinations thereof.

119. Apparatus according to claim 117, wherein when there is one user-actuated device, said device is multifunctional and capable of separately marking occurrence of a plurality of different events of a predetermined nature.

120. Apparatus according to claim 117, wherein when there is a plurality of user-actuated devices, each of said devices is dedicated to marking single or repetitive occurrence of an event of a predetermined nature.

121. Apparatus according to claim 117, wherein said marker means is capable of determining a relative intensity of each occurrence of said patient-subjectively-experienced event relating to bladder function triggering user actuation of said marker means.

122. Apparatus according to claim 121, wherein relative intensity of each said occurrence of said patient-subjectively-experienced event relating to bladder function is expressed through a characteristic parameter of said marker means.

123. Apparatus according to claim 122, wherein said marker means is a variable rheostat and characteristic parameter is electrical resistance.

124. Apparatus according to claim 121, wherein a parameter of an output signal of said marker means is proportional to a relative intensity of said patient-subjectively-experienced event relating to bladder function.

125. Apparatus according to claim 124, wherein:
when said marker means includes a button, relative intensity of said patient-subjectively-experienced event relating to bladder function is expressed by one of proportionality of a user-applied force to said button and frequency of repetitive actuation of said button;
when said marker means includes a switch, relative intensity of said patient-subjectively-experienced event relating to bladder function is expressed by one of selection of a position of a more than bipositional multipositional switch and by frequency of repetitive actuation of said switch; and
when said marker means includes a rotatable knob, relative intensity of said patient-subjectively-experienced event relating to bladder function is expressed by selection of a position of said knob coordinated to indicia of intensity.

126. Apparatus according to claim 116, wherein said patient-subjectively-experienced event relating to bladder function is selected from the group consisting of: consumption of liquid by said patient; sensation of an urge to urinate; and urination.

127. Apparatus according to claim 115, further comprising video image recording means for recording patient image data.

128. Apparatus according to claim 127, wherein said video image recording means comprises one selected from the group consisting of a video camera and a digital camera.

129. Apparatus according to claim 127, wherein said video image recording means is used to record patient image data, while said patient is sleeping.

130. Apparatus according to claim 129, wherein said patient image data recorded while said patient is sleeping is used to determine occurrence of events symptomatic of impaired bladder function in said patient.

131. Apparatus according to claim 128, wherein said video image recording means is capable of recording patient image data for a period of up to 12 hours.

132. Apparatus according to claim 128, wherein when said video image recording means is a digital camera, said digital camera further comprises digital storage means for storing digital image data recorded by said digital camera for a period of up to 12 hours.

133. Apparatus according to claim 132, wherein said digital storage means comprises at least one digital memory card.

134. Apparatus according to claim 127, further comprising a transmitter for transmitting data representative of measured values obtained by at least some of said measuring means and said detection means.

135. Apparatus according to claim 134, wherein said transmitter is electronic and said data is transmitted electronically.

136. Apparatus according to claim 134, further comprising a receiver for receiving data transmitted by said transmitter.

137. Apparatus according to claim 136, wherein said receiver is electronic and said data is transmitted by said transmitter and received by said receiver electronically.

138. Apparatus according to claim 137, wherein said data is electronically transmitted and received by one selected from the group consisting of: radio waves; and light waves.

139. Apparatus according to claim 138, wherein said data is electronically transmitted and received by radio waves.

140. Apparatus according to claim 140, wherein said radio waves are short range radio waves.

141. Apparatus according to claim 137, wherein electronic data transmission and reception is wireless.

142. Apparatus according to claim 137, wherein at least one of electronic data transmission by said transmitter; electronic data reception by said receiver; and electronic communication between said transmitter and said receiver, occurs automatically.

143. Apparatus according to claim 136, wherein said data recording and storing means and said receiver are proximally located.

144. Apparatus according to claim 136, wherein said data recording and storing means is housed in a portable module.

145. Apparatus according to claim 144, wherein said portable module is patient-wearable.

146. Apparatus according to claim 144, wherein all of said measuring means and said detection means are in electronic communication with said module.

147. Apparatus according to claim 146, wherein said module is battery powered.
